(19) United States
(12) Patent Application Publication
(10) Pub. No.: US 2020/0352459 A1
(43) Pub. Date: Nov. 12, 2020

(54) WEARABLE THERMOELECTRIC DEVICES

(71) Applicant: 3M INNOVATIVE PROPERTIES COMPANY, St. Paul, MN (US)

(72) Inventor: Jae Yong Lee, Santa Clara, CA (US)

(21) Appl. No.: 16/641,530

(22) PCT Filed: Aug. 9, 2018
(86) PCT No.: PCT/IB2018/056016

§ 371 Date: Feb. 24, 2020

Related U.S. Application Data
(60) Provisional application No. 62/552,671, filed on Aug. 31, 2017.

(51) Int. Cl. A61B 5/04; H01L 35/32; H04M 1/725; H04W 4/80

(52) U.S. Cl. CPC A61B 5/04; H04W 4/80; H04M 1/7253; H01L 35/32

(57) ABSTRACT

Wearable thermoelectric devices are provided for spatial and temporal cooling/heating on multiple spots of a wearer's skin. The devices include multiple thermoelectric units supported by a flexible band as an array. A control circuit provides switch signals to selectively and sequentially turn on and turn off the thermoelectric units.

### TECHNICAL FIELD

The present disclosure relates to wearable thermoelectric devices that can be utilized for spatial and temporal cooling/heating, and methods of making and using the same.

### BACKGROUND

Wearable devices are widely available for various functions. For example, smart wristbands or watches can be used as activity trackers.

### SUMMARY

There is a desire to deliver personal comfort for a wearer by controlling and managing temperature on multiple spots of the wearer's skin. The present disclosure provides wearable thermoelectric devices that can be utilized for spatial and temporal cooling/heating on multiple spots of the wearer's skin.

In one aspect, the present disclosure describes a wearable thermoelectric device including a flexible band having an inner side and an outer side opposite to the inner side. The device further includes a plurality of thermoelectric units supported by the flexible band as an array, each thermoelectric unit including a working surface on the inner side of the flexible band. A control circuit is electrically connected to the array of thermoelectric units. The control circuit is configured to provide switch signals to the array of thermoelectric units to selectively and sequentially turn on and turn off the thermoelectric units.

In another aspect, the present disclosure describes a system including a wearable thermoelectric device. The device includes a flexible band having an inner side and an outer side opposite to the inner side; a plurality of thermoelectric units supported by the flexible band as an array, each thermoelectric unit including a working surface on the inner side of the flexible band; and a control circuit electrically connected to the array of thermoelectric units. The control circuit is configured to provide switch signals to the array of thermoelectric units to selectively and sequentially turn on and turn off the thermoelectric units. A graphical user interface (GUI) is executed by a processor, and configured to receive instructions from a user, and send the instructions to the control circuit of the device.

In another aspect, the present disclosure describes a method including providing a plurality of thermoelectric units supported by a flexible band as an array. Each thermoelectric unit includes a working surface on an inner side of the flexible band. The method further includes providing switch signals, via a control circuit, to the array of thermoelectric units to selectively and sequentially turn on and turn off the thermoelectric units.

Various unexpected results and advantages are obtained in exemplary embodiments of the disclosure. One such advantage of exemplary embodiments of the present disclosure is that the wearable thermoelectric devices described herein can provide various cooling/heating paths/patterns on multiple spots of a wearer's skin. The performance of the wearable thermoelectric devices can be manually controlled by the wearer or automatically customized to a designated wearer.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

The disclosure may be more completely understood in consideration of the following detailed description of various embodiments of the disclosure in connection with the accompanying figures, in which:

FIG. 1 illustrates a perspective side view of a wearable thermoelectric device, according to one embodiment.

FIG. 2A illustrates a schematic diagram of a wearable thermoelectric device, according to one embodiment.

FIG. 2B illustrates a schematic diagram of a portion of wearable thermoelectric device, according to one embodiment.

FIG. 3A illustrates a top view of a wearable thermoelectric device, according to one embodiment.

FIG. 3B illustrates a cross-sectional view of the wearable thermoelectric device of FIG. 3A.

FIG. 4 illustrates a schematic diagram of cooling/heating paths of a wearable thermoelectric device, according to some embodiments.

FIG. 5 illustrates a schematic diagram of a wearable thermoelectric device wirelessly connected to a mobile device, according to one embodiment.

FIG. 6 illustrates an exemplary screenshot of graphical user interface (GUI) for receiving instructions from a user, and controlling a wearable thermoelectric device, according to one embodiment.

FIG. 7A is an exemplary screenshot of a portion of the GUI of FIG. 6, according to one embodiment.

FIG. 7B is an exemplary screenshot of a portion of the GUI of FIG. 6, according to another embodiment.

FIG. 7C is an exemplary screenshot of a portion of the GUI of FIG. 6, according to another embodiment.

FIG. 8A illustrates a portion of GUI provided for a wearable thermoelectric device, according to one embodiment.

FIG. 8B illustrates a portion of GUI provided for a wearable thermoelectric device, according to another embodiment.

FIG. 8C illustrates a portion of GUI provided for a wearable thermoelectric device, according to another embodiment.

FIG. 8D illustrates a portion of GUI provided for a wearable thermoelectric device, according to another embodiment.

FIG. 8E illustrates a portion of GUI provided for a wearable thermoelectric device, according to another embodiment.

FIG. 8F illustrates a portion of GUI provided for a wearable thermoelectric device, according to another embodiment.

FIG. 8G illustrates a portion of GUI provided for a wearable thermoelectric device, according to another embodiment.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

The present disclosure provides wearable thermoelectric devices that can be utilized for spatial and temporal cooling/heating on multiple spots of a wearer's skin. There are many occasions where people may feel uncomfortable due to ambient environment, such as humidity, high temperature, strong direct sunlight, indoor air conditioning, sweat, etc. The wearable thermoelectric devices described herein can deliver personal comfort for a wearer by controlling and managing temperature on multiple spots on the wearer's skin, for instance, the wrist area. The devices and methods described herein can be extensively applied to various locations such as, for example, a wrist, an arm, a forehead, an area beneath ears, etc. The devices and methods described herein can be applied alone or together with various personal safety products.

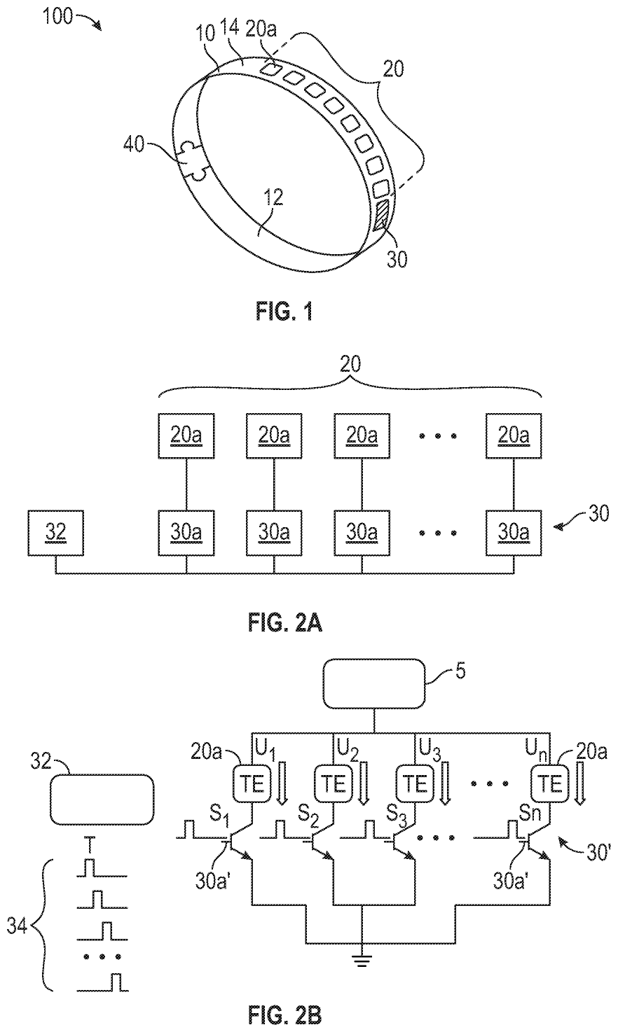

FIG. 1 is a side perspective view of a wearable thermoelectric device 100, according to one embodiment. The wearable thermoelectric device 100 includes a flexible band 10 having an inner side 12 and an outer side 14 opposite to the inner side 12. When the wearable thermoelectric device 100 is disposed on a wearer, the inner side 12 can be in direct contact with the wearer's skin and the outer side 14 can be exposed to air.

An array 20 of thermoelectric units 20a are supported by the flexible band 10. Each thermoelectric unit 20a includes a working surface on the inner side 12 of the flexible band 10. When wearable thermoelectric device 100 is worn on the body of a wearer, the working surfaces of the thermoelectric units 20a can be in direct contact or close proximate to multiple spots on the wearer's body. It is to be understood that the flexible band can be configured, shaped or structured according to applications on various body locations such as, for example, a wrist, an arm, a forehead, an area beneath ears, etc.

The device 100 further includes a control circuit 30 supported by the flexible band 10. The control circuit 30 is electrically connected to the array 20 of thermoelectric units 20a. The control circuit 30 is configured to provide switch signals to the array 20 of thermoelectric units 20a to selectively and sequentially turn on and turn off the thermoelectric units 20a.

The wearable thermoelectric device 100 as depicted in FIG. 1 is a flexible wristband. The flexible band 10 can be made of any suitable flexible materials such as, for example, a woven fabric, a nonwoven fabric, a polymeric material, etc. A mechanical clip 40 is provided to connect opposite ends of the flexible band 10 such that the working surfaces of the thermoelectric units 20a can be pressed against the wearer's body surface.

FIG. 2A illustrates a schematic diagram of the control circuit 30 for controlling the array 20 of thermoelectric units 20a, according to one embodiment. The control circuit 30 includes an array of switches 30a electrically connected to the thermoelectric units 20a and a microprocessor 32 electrically connects to the switches 30a. The microprocessor 32 can generate and send control signals to the array of switches 30a to selectively and sequentially turn on and turn off the thermoelectric units 20a.

In the depicted embodiment of FIG. 2A, the switches 30a are connected in parallel to control the corresponding thermoelectric units 20a. In some embodiments, the switches 30a can simultaneously receive control signals from the microprocessor 32. The control signals can include a series of “on” and “off” signals. When a control signal received by a switch is a “on” signal, the corresponding thermoelectric unit can be turned on; when a control signal received by a switch is an “off” signal, the corresponding thermoelectric unit can be turned off. In this manner, the microprocessor 32 can send various “on” and “off” signals to the switches 30a to selectively and sequentially turn on and turn off the thermoelectric units 20a.

In the depicted embodiment of FIG. 2B, the microprocessor 32 can generate sequential pulse signals 34 to selectively and sequentially turn on and turn off the thermoelectric units 20a which are powered by a power source 5. The pulse signals 34 can selectively turn on one or more of the thermoelectric units and turn off the one or more thermoelectric units after a time period T. For example, at time t₁, the microprocessor 32 can send a pulse signal to the switch s₁ to turn on the thermoelectric unit u₁ connected to the switch s₁ and turn off the thermoelectric unit u₁ after a time period T; at time t₂, the microprocessor 32 can send a pulse signal to the switch s₂ to turn on the thermoelectric unit u₂ connected to the switch s₂ and turn off the thermoelectric unit u₂ after a time period T; at time t₃, the microprocessor 32 can send a pulse signal to the switch s₃ to turn on the thermoelectric unit u₃ connected to the switch s₃ and turn off the thermoelectric unit u₃ after a time period T; and at time tₙ, the microprocessor 32 can send a pulse signal to the switch s₁₁ to turn on the thermoelectric unit u₁₁ connected to the switch s₁₁ and turn off the thermoelectric unit u₁₁ after a time period T. It is to be understood that an “on/off” signal (e.g., a pulse signal) can be sent to multiple switches at the same time to turn on/off the corresponding thermoelectric units. It is also to be understood that the time period T can be variable. The time period T can also be controlled, via the control circuit 30, to be the same or different for different thermoelectric units and/or at different time.

The control circuit 30′ of FIG. 2B includes transistors 30a′ as switches. It is to be understood that any suitable switches other than transistors can be used to turn on and turn off the thermoelectric units 20a. It is also to be understood that the microprocessor 32 can generate any suitable control signals other than the pulse signals 34 to control the switches so as to selectively and sequentially turn on/off the thermoelectric units 20a.

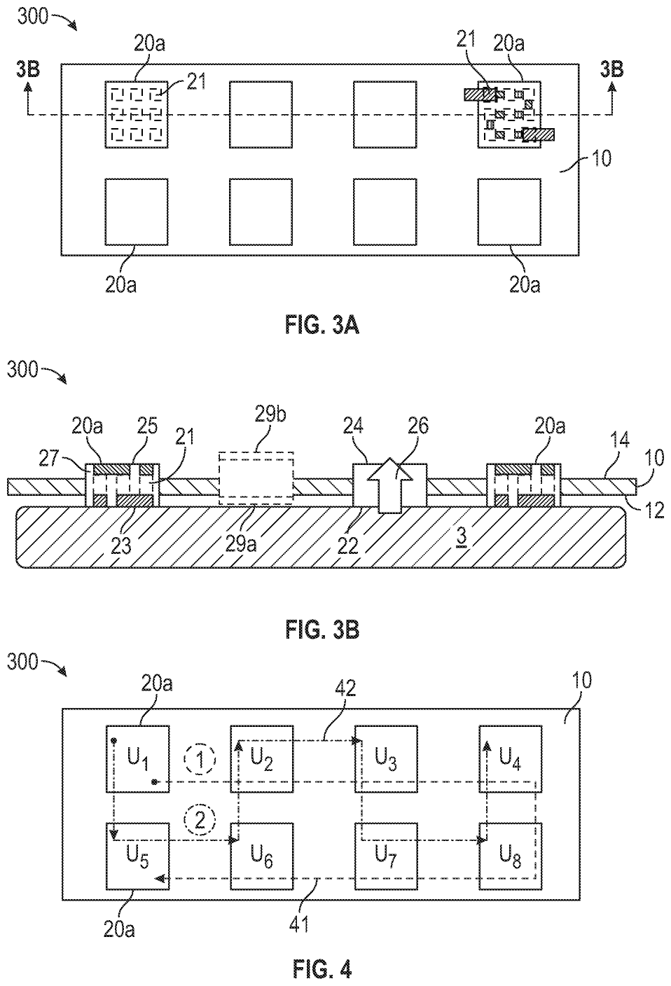

FIG. 3A illustrate a top portion view of a wearable thermoelectric device 300, according to one embodiment. FIG. 3B illustrate a cross sectional view of the wearable thermoelectric device 300. The wearable thermoelectric device 300 includes the flexible band 10 and an array of thermoelectric units 20a supported by the flexible band 10. The thermoelectric units 20a each include one or more thermoelectric p-n junctions 21. In some embodiments, the thermoelectric p-n junctions 21 can be formed by electrically connecting an array of n-type and p-type thermoelectric chips to form a thermoelectric circuit. The thermoelectric chips can be supported by a flexible substrate 27 connected to the flexible band 10. In some embodiments, the flexible substrate 27 can be stitched or glued along the rim to the flexible band 10. Exemplary flexible thermoelectric units or modules and methods of making the thermoelectric units or modules are the described in PCT/US2017/038690 (Lee et al.), which is incorporated herein by reference.

In the depicted embodiment of FIG. 3B, each thermoelectric unit 20a includes an array of n-type and p-type thermoelectric chips. The respective ends of the chips are electrically connected by electrodes 23 and 25 to form a thermoelectric circuit (e.g., the thermoelectric p-n junctions 21). When an electric current flow in the thermoelectric circuit, a temperature difference between the opposite surfaces 22 and 24 of the thermoelectric unit 20a can be generated via a heat flow 26.

The wearable thermoelectric device 300 can be disposed on an object surface 3 (e.g., a human skin), with the inner side 12 facing the object surface 3. The working surfaces 22 of the thermoelectric units 20a can be in direct contact or close proximate with multiple spots on the object surface 3. In some embodiments, the working surface 22 of the thermoelectric units 20a may have a contacting area in the range of, for example, from about 5 mm² to about 10 cm², from about 10 mm² to about 10 cm², or from about 25 mm² to about 5 cm². The distance between the adjacent thermoelectric units 20a may be in the range of, for example, from about 1 mm to about 10 cm, from about 2 mm to about 5 cm, or from about 5 mm to about 2 cm. The multiple spots on the object surface 3 in contact with the thermoelectric units 20a may be discrete, separate from each other.

In the depicted embodiment, the working surfaces 22 each are a cooling surface, and the thermoelectric units 20a each are configured to allow the heat flow 26 from the respective cooling surfaces 22 to the hot surfaces 24 thereof on the outer side 14 of the flexible band 10. It is to be understood that in some embodiments, the working surfaces 22 can be a heating surface and the outer surfaces 24 can be a cool surface; in some embodiments, the working surfaces 22 of some thermoelectric units 20a can be a cooling surface and the working surfaces 22 of other thermoelectric units 20a can be a heating surface. The arrangement of the thermoelectric units 20a to cool or heat multiple spots on the object surface 3 may depend on desired applications.

Optionally, a thermally conductive layer 29a can be disposed between the working surface 22 and the object surface 3 to promote the heat exchange therebetween. The thermally conductive layer 29a may include any suitable thermally conductive materials such as, for example, Carbon Nanotubes (CNTs)-based composites. In some embodiments, the thermally conductive layer 29a may include a non-adhesive thermal conductive surface to be in contact with a skin. The thermally conductive layer 29a may include a thermally conductive acrylic interface pad, or a thermally conductive silicone interface pad, commercially available from 3M Company (Saint Paul, Minn., USA).

The hot/cool surfaces 24 are exposed to air for heat exchange. In some embodiments, the wearable thermoelectric device 300 may include an optional thermally conductive layer 29b disposed on the hot surfaces 24 of the thermoelectric unit 20a. The optional thermally conductive layer 29b may include, for example, Super Absorbent Polymers (SAP) with Metal Organic Frameworks (MOF). In some embodiments, the thermally conductive layer 29b may include a heat spreading layer such as, for example, a thermally conductive heat spreading tape commercially available from 3M Company (Saint Paul, Minn., USA).

The wearable thermoelectric device described herein can selectively and sequentially turn on and turn off the thermoelectric units thereof to form various cooling/heating paths/patterns on multiple spots of an object surface for spatial and temporal cooling/heating. FIG. 4 illustrates a schematic diagram of exemplary cooling/heating paths 41 and 42 of the wearable thermoelectric device 300, according to some embodiments. In the cooling/heating path 41, the thermoelectric units 20a are turned on and off sequentially in this order: u₁ on and off, u₂ on and off, u₃ on and off, u₄ on and off, u₈ on and off, u₇ on and off, u₆ on and off, and u₅ on and off In the cooling path 42, the thermoelectric units 20a in the first and second rows are sequentially turned on and turned off in this order: u₁ on and off, u₅ on and off, u₆ on and off, u₂ on and off, u₃ on and off, u₇ on and off, u₆ on and off, and u₄ on and off.

The cooling/heating paths/patterns described herein may refer to a time and spatial distribution of the on/off states of the thermoelectric units. It is to be understood that various cooling/heating paths/patterns can be formed by selectively and sequentially turn on and turn off the thermoelectric units, via a control circuit to provide switch signals to the thermoelectric units. In the embodiment of FIG. 4, the thermoelectric units are sequentially turn on and off one by one. It is to be understood that multiple thermoelectric units can be selected and turned on at the same time, while other thermoelectric units are in an “off” state. It is also to be understood that the sequentially turned-on thermoelectric units do not have to be spatially adjacent to each other.

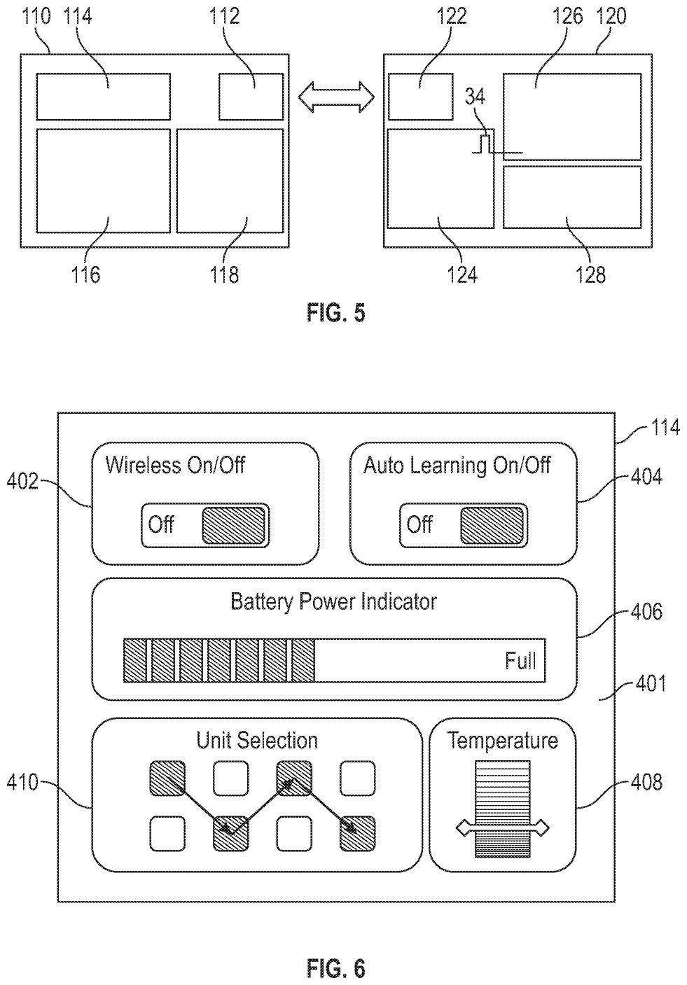

FIG. 5 illustrates a schematic diagram of a wearable thermoelectric device 120 wirelessly connected to a mobile device 110, according to one embodiment. The wearable thermoelectric device 120 includes an array of thermoelectric units 124, and a control circuit 126 electrically connected to the thermoelectric units 124 to selectively and sequentially turn on/off (e.g., via the pulse signal 34) the thermoelectric units 124. The wearable thermoelectric device 120 further includes a wireless component 122 connected to the control circuit 126. The wireless component 122 may include, for example, a Bluetooth Low Energy (BLE) component.

The mobile device 110 includes a wireless component 112 that can work with the wireless component 122 of the wearable thermoelectric device 120 for data transmission between the mobile device 110 and the wearable thermoelectric device 120. The mobile device 110 further includes a graphical user interface (GUI) 114 that is executed by a processor 116 and displayed by a display 118 thereof.

In some embodiments, the GUI 114 can be provided as a mobile app that runs on the mobile device 110, e.g., a smart phone. The mobile app can be a computer program in any suitable programming language (e.g., Python) designed to be executed by the processor 116.

The processor 116 may include, for example, one or more general-purpose microprocessors, specially designed processors, application specific integrated circuits (ASIC), field programmable gate arrays (FPGA), a collection of discrete logic, and/or any type of processing device capable of executing the techniques described herein.

The mobile device may also include a memory to store information. The memory can store instructions for forming the methods or processes (e.g., a machine learning algorithm) described herein. The memory can also store data related to the wearable thermoelectric device.

It is to be understood that in some embodiments, the mobile device can be integrated with the wearable thermoelectric device to be a single device in the form of, for example, a smart watch.

FIG. 6 is a screenshot of the exemplary graphical user interface (GUI) 114 for receiving instructions from a user for manually or automatically controlling a wearable thermoelectric device, according to one embodiment. The GUI 114 includes a GUI screen 401 within which a user can input instructions and view information related to the wearable thermoelectric device 120. Within the GUI screen 401, a wireless button 402 is provided for a user to use it to turn on/off a wireless connection (e.g., a wireless connection between the wireless components 112 and 122). An auto-learning button 404 is provided for the user to use it to turn on/off auto-learning functions which will be discussed further below. A battery power box 406 is provided to indicate a power level of the power source 128 of the wearable thermoelectric device 120. A temperature box 408 is provided for displaying temperature information of the thermoelectric units and/or for a user to use it to adjust the temperatures of the thermoelectric units. A unit selection box 410 that a user can use to create cooling/heating paths or patterns. It is to be understood that the GUI 114 can include any suitable functional buttons, boxes or other tools that a user can use to view, monitor, and control the operation of the wearable thermoelectric device. The functions of the auto-learning button 404 and the unit selection box 410 will be further described below.

When the auto-learning button 404 is turned on, a processor (e.g., the processor 116 of FIG. 5) can execute a machine learning algorithm to automatically optimize the operation of a wearable thermoelectric device. The processor can execute program instructions (e.g., software instructions) to carry out the machine learning algorithm described herein. Such optimization can be based on available data such as, for example, preference, ambient conditions, previous operation history, etc.

In some embodiments, when the auto-learning button 404 is turned on, the machine learning algorithm can monitor a user's operation of the GUI to control the wearable thermoelectric device. For example, the machine learning algorithm can track down cooling/heating paths or patterns that a user creates via the unit selection box 410.

In some embodiments, the machine learning algorithm can further analyze the monitored data, determine the user preferred cooling/heating paths or patterns under certain circumstances based on the analysis, and generate decision-making data. The decision-making data can be sent to a control circuit of the wearable thermoelectric device (e.g., the control circuit 126 of FIG. 5) to run the wearable thermoelectric device in an optimized or user preferred manner.

In some embodiments, the machine learning algorithm can automatically generate decision-making data to run the wearable thermoelectric device. The decision-making data can be generated by applying data related to various reference parameters such as, for example, environmental references/factors, and data related to history references (e.g., previous user selected modes). The environmental references/factors may include, for example, local weather conditions (e.g., temperature, humidity, wind, etc.), an amount of user's activity (e.g., data from an accelerometer), local time and location information (e.g., city, beach, gym, etc.), ambient light (e.g., outdoor, indoor, day, or night), etc.

In some embodiments, the machine learning algorithm can generate decision-making data by correlating environmental factors and history references. The generated decision-making data can represent the most optimized operation mode for the best feeling performance customized solely to a designated user. The wearable thermoelectric device described herein, when coupled with the machine learning algorithm, can provide a wearer with compelling comfort by learning what the wearer really wants regarding a cooling/heating of multiple spots on the wearer's skin.

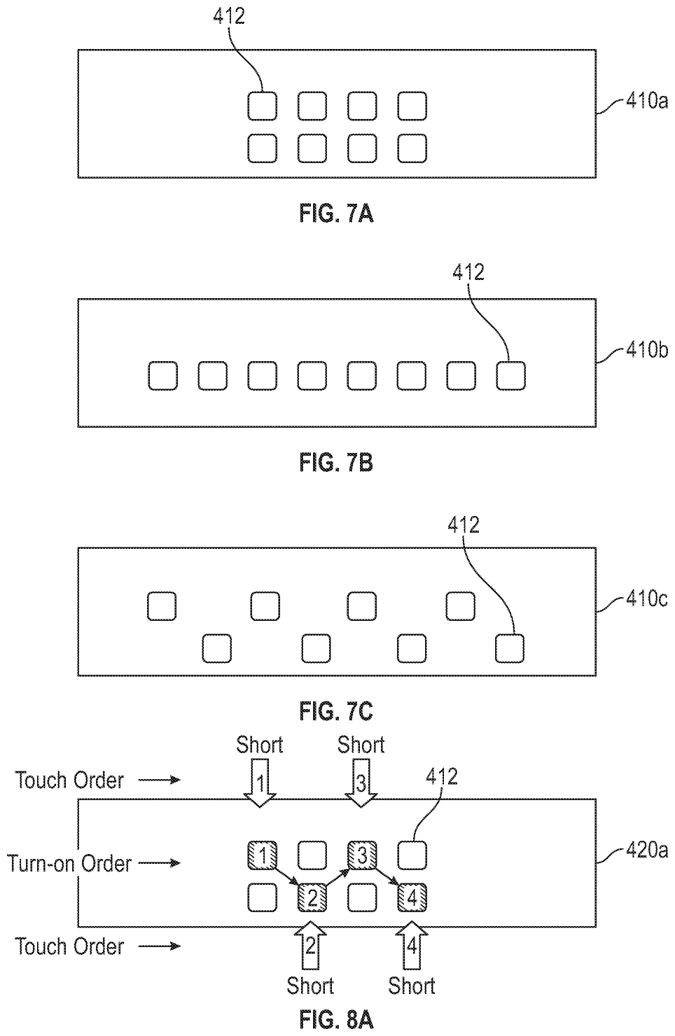

FIGS. 7A-C are screenshots of various exemplary unit selection boxes 410a, 410b and 410c provided by a GUI (e.g., the GUI 114 of FIG. 5) that a user can use to create cooling/heating paths or patterns. The unit selection boxes each include multiple buttons/tools 412 which represent an array of thermoelectric units of a wearable thermoelectric device (e.g., the thermoelectric units 124 of FIG. 5). The buttons 412 are selectable such that a user can select one or more of the buttons 12 to create a cooling/heating path or pattern. As shown in FIGS. 7A-C, the buttons 412 can be arranged in various configurations. In some embodiments, the arrangement of buttons 412 in the unit selection boxes can track the arrangement of the thermoelectric units 124 on the wearable thermoelectric device.

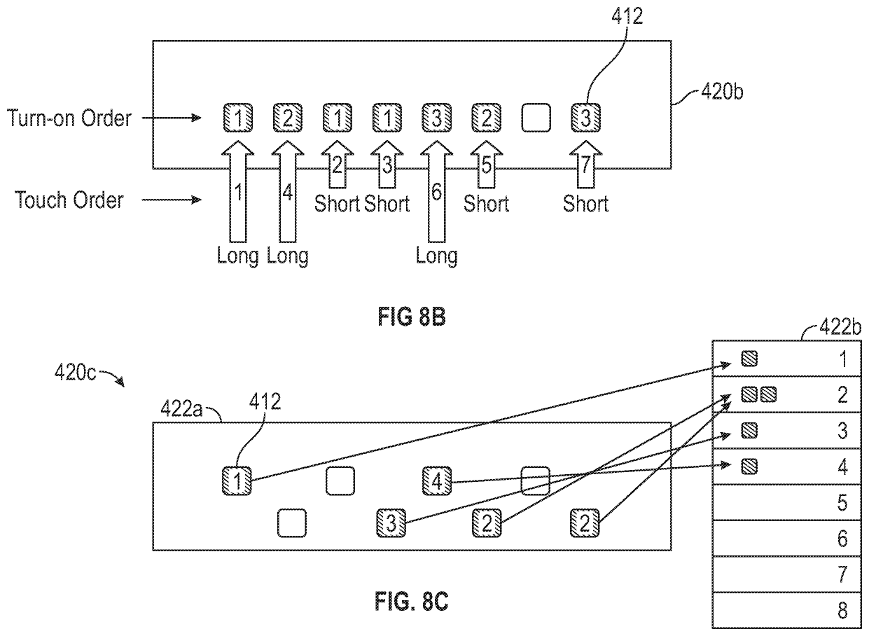

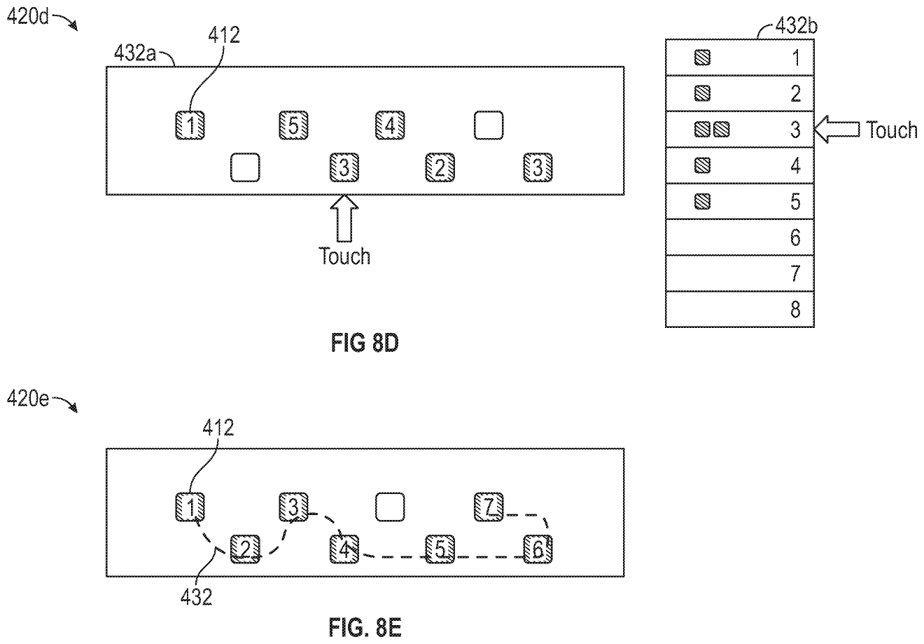

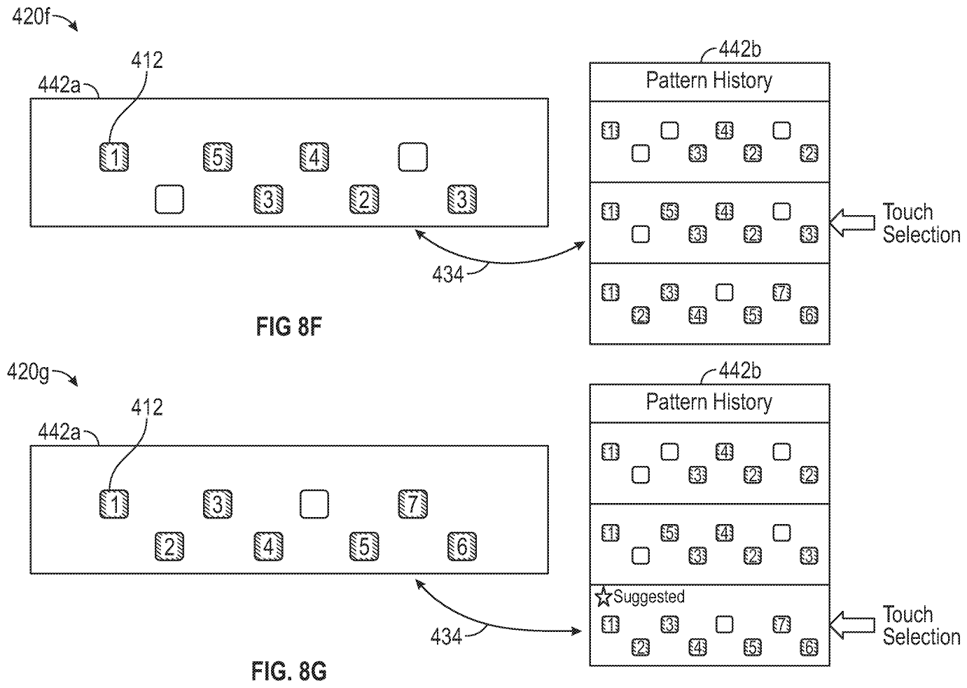

FIGS. 8A-G are screenshots of various exemplary unit selection boxes provided by a GUI with which a user can use to create cooling/heating paths or patterns. In the unit selection box 420a of FIG. 8A, some of the buttons 412 are selected (e.g., by a short touching via fingers) in the sequence of 1, 2, 3 and 4 to form a cooling/heating path or pattern.

In the unit selection box 420b of FIG. 8B, some of the buttons 412 are selected (e.g., by a short or long touching) in the sequence of 1, 2, 3, 4, 5, 6 and 7, where 1, 4, and 6 are respectively selected by a long touching, and 2, 3, 5 and 7 are selected by a short touching. In this manner, the buttons 412 are grouped into sequential groups (e.g., groups 1, 2 and 3), where the corresponding groups of thermoelectric units can be controlled to be sequentially turned on/off.

The unit selection box 420c of FIG. 8C includes a first sub-box 422a which includes the array of buttons 412, and a second sub-box 422b to group the buttons 412 and sequentially list the groups. In the depicted embodiment, some of the buttons 412 in the first sub-box 422a are selected by dragging and dropping the respective buttons 412 into a second sub-box 422b, where the buttons are grouped and listed in a sequential order (e.g., 1, 2, 3 . . . ) to form a cooling/heating path or pattern.

The unit selection box 420d of FIG. 8D includes a first sub-box 432a which includes the array of buttons 412, and a second sub-box 432b to group the buttons 412 and sequentially list the groups. In the depicted embodiment, one of the groups in the second sub-box 432b (e.g., groups 1, 2, 3 . . . 8) can be selected (e.g., by touching) first, and the corresponding buttons 412 in the first sub-box 432a can be selected and grouped into that selected group. A group label (e.g., 1, 2, 3, 4, 5) corresponding to the group number can be shown for the selected buttons 412 in the first sub-box 432a.

In the unit selection box 420e of FIG. 8E, some of the buttons 412 are selected, via a draw by touch-swiping in the sequence of 1, 2, 3, 4, 5, 6 and 7. A directional wire 432 is shown in the box 420 to sequentially connect the selected buttons 412. In this manner, the corresponding thermoelectric units can be controlled to be sequentially turned on/off in the order shown by the directional wire 432.

The unit selection box 420f of FIG. 8F includes a first sub-box 442a which includes the array of buttons 412, and a second sub-box 442b which lists available options of various cooling/heating paths or patterns. The cooling/heating path or pattern in the second sub-box 442b can be selected (e.g., by a finger touching) and/or dragged, via a path 434, into the first sub-box 442. The selected cooling/heating path or pattern can be shown in the first sub-box 442a. In some embodiments, the cooling/heating paths or patterns listed in the second sub-box 442b can be labeled. For example, as shown in FIG. 8G, one of the cooling/heating paths is labeled as “suggested,” which can be selected by the machine learning algorithm discussed above.

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

### Listing of Exemplary Embodiments

Exemplary embodiments are listed below. It is to be understood that any one of embodiments 1-13 and 14-24 can be combined.

Embodiment 1 is a wearable thermoelectric device comprising:

a flexible band having an inner side and an outer side opposite to the inner side;

a plurality of thermoelectric units supported by the flexible band as an array, each thermoelectric unit including a working surface on the inner side of the flexible band; and

a control circuit electrically connected to the array of thermoelectric units,

wherein the control circuit is configured to provide switch signals to the array of thermoelectric units to selectively and sequentially turn on and turn off the thermoelectric units.

Embodiment 2 is the device of embodiment 1, wherein at least one of the working surfaces is a cooling surface.
Embodiment 3 is the device of embodiment 1 or 2, wherein the thermoelectric units each include one or more thermoelectric p-n junctions.
Embodiment 4 is the device of embodiment 2 or 3, wherein at least one of the thermoelectric units includes a hot surface exposed to air.
Embodiment 5 is the device of any one of embodiments 1-4, further comprising a thermally conductive layer disposed on the working surface.
Embodiment 6 is the device of any one of embodiments 1-5, wherein the control circuit further comprises a microprocessor and a plurality of switches, the microprocessor is configured to generate sequential pulse signals to the plurality of switches.
Embodiment 7 is the device of embodiment 6, wherein the switches are connected in parallel.
Embodiment 8 is the device of embodiment 6 or 7, wherein the switches each include a transistor.
Embodiment 9 is the device of any one of embodiments 1-8 further comprises a wireless component connected to the control circuit.
Embodiment 10 is the device of embodiment 9, wherein the wireless component includes a Bluetooth Low Energy (BLE) component.
Embodiment 11 is the device of any one of embodiments 1-10 being a wristband which further comprises a mechanical clip to connect opposite ends thereof.
Embodiment 12 is a system comprising:

the wearable thermoelectric device of any one of the preceding embodiments; and

a graphical user interface (GUI) executed by a processor, the GUI configured to receive instructions from a user, and send the instructions to the control circuit of the device.

Embodiment 13 is the system of embodiment 12, wherein the GUI is provided to a mobile device. Embodiment 14 is a method comprising:

providing a plurality of thermoelectric units supported by a flexible band as an array, each thermoelectric unit including a working surface on an inner side of the flexible band; and

providing switch signals, via a control circuit, to the array of thermoelectric units to selectively and sequentially turn on and turn off the thermoelectric units.

Embodiment 15 is the method of embodiment 14, wherein the working surface is a cooling surface, and when one or more of the thermoelectric units each are selected and turned on, the selected thermoelectric units are configured to allow a heat flow from the respective cooling surfaces to one or more hot surfaces thereof on an outer side of the flexible band.
Embodiment 16 is the method of embodiment 15, further comprising exposing the hot surfaces of the thermoelectric units to air.
Embodiment 17 is the method of embodiment 15 or 16, further comprising providing a thermally conductive layer disposed on the hot surfaces of the thermoelectric units.
Embodiment 18 is the method of any one of embodiments 14-17, wherein providing the switch signals further comprises generating, via a microprocessor, sequential pulse signals to a plurality of switches connected to the plurality of thermoelectric units.
Embodiment 19 is the method of any one of embodiments 14-18 further comprises providing a wireless component connected to the control circuit.
Embodiment 20 is the method of any one of embodiments 14-19 further comprising receiving, via a graphical user interface (GUI) executed by a processor, instructions from a user, and sending the instructions to the control circuit.
Embodiment 21 is the method of any one of embodiments 14-20, wherein providing the switch signals further comprises automatically generating decision-making data, via a machine learning algorithm, and sending the generating decision-making data to the control circuit to generate the switch signals.
Embodiment 22 is the method of embodiment 21, wherein the decision-making data are generated by correlating environmental factors and history references.
Embodiment 23 is the system of embodiments 12 or 13, wherein the GUI provides multiple selectable buttons which represent the array of thermoelectric units of the wearable thermoelectric device.
Embodiment 24 is the method of embodiment 20, wherein the GUI provides multiple selectable buttons which represent the array of thermoelectric units of the wearable thermoelectric device.

Reference throughout this specification to “one embodiment,” “certain embodiments,” “one or more embodiments,” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus, the appearances of the phrases such as “in one or more embodiments,” “in certain embodiments,” “in one embodiment,” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments.

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. In particular, as used herein, the recitation of numerical ranges by endpoints is intended to include all numbers subsumed within that range (e.g., 1 to 5 includes 1, 1.5, 2, 2.75, 3, 3.80, 4, and 5). In addition, all numbers used herein are assumed to be modified by the term “about.” Furthermore, various exemplary embodiments have been described. These and other embodiments are within the scope of the following claims.

1. A wearable thermoelectric device comprising:
a flexible band having an inner side and an outer side opposite to the inner side;
a plurality of thermoelectric units supported by the flexible band as an array, each thermoelectric unit including a working surface on the inner side of the flexible band; and
a control circuit electrically connected to the array of thermoelectric units,
wherein the control circuit is configured to provide switch signals to the array of thermoelectric units to selectively and sequentially turn on and turn off the thermoelectric units.

2. The device of claim 1, wherein at least one of the working surfaces is a cooling surface.

3. The device of claim 1, wherein the thermoelectric units each include one or more thermoelectric p-n junctions.

4. The device of claim 2, wherein at least one of the thermoelectric units includes a hot surface exposed to air.

5. The device of claim 1, further comprising a thermally conductive layer disposed on the working surface.

6. The device of claim 1, wherein the control circuit further comprises a microprocessor and a plurality of switches, the microprocessor is configured to generate sequential pulse signals to the plurality of switches.

7. The device of claim 6, wherein the switches are connected in parallel.

8. The device of claim 6, wherein the switches each include a transistor.

9. The device of claim 1 further comprises a wireless component connected to the control circuit.

10. The device of claim 9, wherein the wireless component includes a Bluetooth Low Energy (BLE) component.

11. The device of claim 1 being a wristband which further comprises a mechanical clip to connect opposite ends thereof.

12. A system comprising:
the wearable thermoelectric device of any one of the preceding claims; and
a graphical user interface (GUI) executed by a processor, the GUI configured to receive instructions from a user, and send the instructions to the control circuit of the device.

13. The system of claim 12, wherein the GUI is provided to a mobile device.

14. A method comprising:
providing a plurality of thermoelectric units supported by a flexible band as an array, each thermoelectric unit including a working surface on an inner side of the flexible band; and
providing switch signals, via a control circuit, to the array of thermoelectric units to selectively and sequentially turn on and turn off the thermoelectric units.

15. The method of claim 14, wherein the working surface is a cooling surface, and when one or more of the thermoelectric units each are selected and turned on, the selected thermoelectric units are configured to allow a heat flow from the respective cooling surfaces to one or more hot surfaces thereof on an outer side of the flexible band.

16-17. (canceled)

18. The method of claim 14, wherein providing the switch signals further comprises generating, via a microprocessor, sequential pulse signals to a plurality of switches connected to the plurality of thermoelectric units.

19. The method of claim 14 further comprises providing a wireless component connected to the control circuit.

20. The method of claim 14 further comprising receiving, via a graphical user interface (GUI) executed by a processor, instructions from a user, and sending the instructions to the control circuit.

21. The method of claim 14, wherein providing the switch signals further comprises automatically generating decision-making data, via a machine learning algorithm, and sending the generating decision-making data to the control circuit to generate the switch signals.

22. The method of claim 21, wherein the decision-making data are generated by correlating environmental factors and history references.
